(19) United States
(12) Patent Application Publication
(10) Pub. No.: US 2008/0286757 A1
(43) Pub. Date: Nov. 20, 2008

(54) Method and Apparatus for Identification of Microorganisms Using Bacteriophage

(75) Inventors: G. Scott Gaisford, Denver, CO (US); John H. Wheeler, Boulder, CO (US); Jon C. Rees, Longmont, CO (US); Scott D. Conlin, Boulder, CO (US)

Correspondence Address: PATTON BOGGS LLP, 1801 CALFORNIA STREET, SUITE 4900, DENVER, CO, 80202, US

(73) Assignee: MicroPhage Incorporated, Longmont, CO (US)

(21) Appl. No.: 12/066,806

(22) PCT Filed: Sep. 15, 2006
(86) PCT No.: PCT/US06/36070

§ 371 Date: Mar. 13, 2008

Related U.S. Application Data
(60) Provisional application No. 60/717,423, filed on Sep. 15, 2005.

(51) Int. Cl. C12Q 1/70

(52) U.S. Cl. 435/5

(57) ABSTRACT

A sample is tested for the presence of bacteria, such as in an automatic blood culturing apparatus. If bacteria are determined to be present, a bacteriophage-based bacteria identification process is performed to identify the bacteria present. A plurality of bacteria detection processes, such as a blood culture test and Gram stain test may be carried out prior to the bacteria identification process. A bacteriophage-based antibiotic resistance test or antibiotic susceptibility test is also conducted on the sample.

### BACKGROUND OF THE INVENTION

1. Field of the Invention

The invention relates generally to the field of identification of microscopic living organisms, and more particularly to the identification of microorganisms using bacteriophage.

2. Statement of the Problem

Standard microbiological methods for identification of microorganisms have relied on substrate-based assays to test for the presence of specific bacterial pathogens. See Robert H. Bordner, John A. Winter, and Pasquale Scarpino, Microbiological Methods For Monitoring The Environment, EPA Report No. EPA-600/8-78-017, US. Environmental Protection Agency, Cincinnati, Ohio, 45268, December 1978. These techniques are generally easy to perform, do not require expensive supplies or laboratory facilities, and offer high levels of selectivity. However, these methods are slow. Substrate-based assays are hindered by the requirement to first grow or cultivate pure cultures of the targeted organism, which can take days. This time constraint severely limits the effectiveness to provide rapid response to the presence of virulent strains of microorganisms.

The long time it takes to identify microorganisms using standard methods is a serious problem resulting in significant human and economic costs. Thus, it is not surprising that much scientific research has been done and is being done to overcome this problem. Some examples are immunomagnetic separation, ELISA, dot blot assay, flow cytometry, and Polymerase Chain Reaction (PCR). However, none of these methods achieve the sensitivity of substrate-based assays, and all are more expensive and typically require more highly trained technicians than do classical substrate-based methods.

Bacteriophage-based methods have been suggested as a method to accelerate microorganism identification. See, for example, U.S. Pat. No. 5,985,596 issued Nov. 16, 1999 and U.S. Pat. No. 6,461,833 B1 issued October 8, both to Stuart Mark Wilson, U.S. Pat. No. 4,861,709 issued Aug. 29, 1989 to Ulitzur et al, U.S. Pat. No. 5,824,468 issued Oct. 20, 1998 to Scherer et al., U.S. Pat. No. 5,656,424 issued Aug. 12, 1997 to Jurgensen et al., U.S. Pat. No. 6,300,061 B1 issued Oct. 9, 2001 to Jacobs, Jr. et al., U.S. Pat. No. 6,555,312 B1 issued Apr. 29, 2003 to Hiroshi Nakayama, U.S. Pat. No. 6,544,729 B2 issued Apr. 8, 2003 to Sayler et al., U.S. Pat. No. 5,888,725 issued Mar. 30, 1999 to Michael F. Sanders, U.S. Pat. No. 6,436,652 B1 issued Aug. 20, 2002 to Cherwonogrodzky et al., U.S. Pat. No. 6,436,661 B1 issued Aug. 20, 2002 to Adams et al., U.S. Pat. No. 5,498,525 issued Mar. 12, 1996 to Rees et al., Angelo J. Madonna, Sheila VanCuyk and Kent J. Voorhees, “Detection Of Esherichia Colil Using Immunomagnetic Separation And Bacteriophage Amplification Coupled With Matrix-Assisted Laser Desorption/Ionization Time-Of-Flight Mass Spectrometry”, Wiley InterScience, DOI:10.1002/rem.900, 24 Dec. 2002, and United States Patent Application Publication No. 2004/0224359 published Nov. 11, 2004. Bacteriophages are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called lytic bacteriophage, rupture the host bacterium releasing the progeny phage into the environment to seek out other bacteria. The total incubation time for infection of a bacterium by parent phage, phage multiplication (amplification) in the bacterium to produce progeny phage, and release of the progeny phage after lysis can take as little as an hour depending on the phage, the bacterium, and the environmental conditions. Thus, it has been proposed that the use of bacteriophage amplification in combination with a test for bacteriophage or a bacteriophage marker may be able to significantly shorten the assay time as compared to a traditional substrate-based identification. However, the above bacteriophage identification assays, in general, have significant problems, such as the need for sophisticated, complicated, lengthy and/or expensive tests to detect the bacteriophage or bacteriophage marker, difficulties associated with distinguishing progeny phage from parent phage, and the fact that strains of bacteriophage that have proven high success in identifying a specific microorganism are not generally available. Thus, despite the promise of shorter time frames to detect microorganisms, no commercially practical phage-based assay has been developed.

Thus, there remains a need for a faster method of detecting microorganisms that achieves the specificity, accuracy and economy of substrate-based methods.

### SUMMARY OF THE INVENTION

The invention solves the above problems, as well as other problems of the prior art by combining ascertaining the presence of a living microorganism in a sample with a process other than a bacteriophage process, and using bacteriophage to identify the microorganism. Preferably, the non-bacteriophage process is performed prior to the bacteriophage process, though it also may be performed in parallel with the bacteriophage process.

Ascertaining the presence of a living microorganism independently of the bacteriophage process solves a number of problems with prior art bacteriophage identification methods. First, if the non-bacteriophage process is done prior to the bacteriophage process, this significantly limits the number of samples on which the bacteriophage process must be performed. Secondly, since bacteriophage identification is inherently much faster than conventional identification processes, several bacteriophage cycles can be performed and the entire process of the invention will still be faster than the conventional substrate culture process. Since, the non-bacteriophage process has already eliminated those samples in which no microorganism is present, the cost of repetitive bacteriophage cycles is both warranted and minimized. The additional cycles increase the reliability of the bacteriophage process. Thirdly, a problem with the accuracy and speed of prior art bacteriophage processes has been the fact that if insufficient numbers of the target microorganism are present, large numbers of parent bacteriophage must be used to be sure the bacteriophage rapidly find the microorganism, which greatly complicates the process of distinguishing progeny bacteriophage. The method of the invention solves this issue because the time during which the non-bacteriophage process is being run can be used to increase the numbers of microorganisms present, which allows a smaller number of parent bacteriophage to be used, which significantly increases the signal to noise ratio of the bacteriophage detection process.

The invention also provides a method of identifying a microorganism present in a sample, said method comprising: (a) performing a test on said sample capable of detecting the presence of a microorganism in said sample without identifying said microorganism; and (b) identifying the microorganism present in said sample using a phage-based microorganism identification process.

In one embodiment, the invention provides a method of identifying a microorganism present in a sample, the method comprising: (a) performing a test on the sample capable of detecting the presence of a microorganism in the sample without identifying the microorganism; (b) if the performing does not detect the presence of a microorganism, declaring a negative result; and (c) if the performing detects the presence of a microorganism in the sample, identifying the microorganism present in the sample using a phage-based microorganism identification process. Preferably, the method further comprises conducting an antibiotic resistance test or antibiotic susceptibility test on the sample. Preferably, the identifying is performed on a first sample, the conducting comprises conducting an antibiotic resistance test on a second sample, and the antibiotic susceptibility test comprises: the identifying the microorganism in the first sample and the conducting the antibiotic resistance test on the second sample. Preferably, the conducting comprises conducting a plurality of antibiotic resistance tests on a plurality of samples, each the antibiotic resistance test utilizing a different antibiotic or a different concentration of antibiotic. Preferably, antibiotic resistance test or the antibiotic susceptibility test comprise a phage-based antibiotic resistance test or a phage-based antibiotic susceptibility test. Preferably, the identifying comprises a calorimetric test. Preferably, the performing comprises carrying out a plurality of different tests capable of detecting the presence of a microorganism in the sample. Preferably, the microorganism is a bacteria and the plurality of different tests are selected from the group consisting of blood culture, autofluorescence, Gram stain, Wright's stain, acridine orange ptl, glucose, dipstick, nitrides-on-silicon chips, microwave resonance cavity, or immunological methods. Preferably, the plurality of tests comprise an automatic blood culture test and a Gram stain test. Preferably, the phage-based microorganism identification process comprises one or more tests selected from the group consisting of: immunoassay methods, aptamer-based assays, mass spectrometry, including MALDI, and flow cytometry. Preferably, the immunoassy methods are selected from the group consisting of ELISA, western blots, radioimmunoassay, immunoflouresence, lateral flow immunochromatography (LFI), and a test using a SILAS surface. Preferably, the microorganism is a bacteria and the performing comprises one or more methods selected from the group consisting of blood culture, autofluorescence, Gram stain, Wright's stain, acridine orange ptl, glucose, dipstick, nitrides-on-silicon chips, microwave resonance cavity, or immunological methods.

In another embodiment, the invention provides a method of identifying a microorganism present in a sample, the method comprising: (a) performing a test on the sample capable of detecting the presence of a microorganism in the sample without identifying the microorganism; and (b) while the performing is being done, identifying the microorganism present in the sample using a phage-based microorganism identification process. Preferably, the method further comprises, if the performing does not detect the presence of a microorganism declaring a negative result.

In another aspect, the invention provides a method of identifying a bacterium present in a sample of blood, said method comprising: (a) combining said sample of blood and a nutrient medium suitable for the growth of bacteria; (b) inserting said at least a first portion of said combined sample in an automatic blood culturing apparatus to determine if bacteria are present in said blood sample; and performing a phage-based microorganism identification process on said first portion or another portion of said combined sample to identify the bacteria present in said blood.

In one embodiment, the invention provides a method of identifying a bacterium present in a sample of blood, the method comprising: (a) combining the sample of blood and a nutrient medium suitable for the growth of bacteria; (b) inserting the combined sample in an automatic blood culturing apparatus to determine if bacteria are present in the blood sample; and (c) if bacteria are determined to be present in the automatic blood culturing apparatus, performing a phage-based microorganism identification process on the combined sample to identify the bacteria present in the blood. Preferably, the method further comprises conducting an antibiotic resistance test or antibiotic susceptibility test on the combined sample. Preferably, the antibiotic resistance test or the antibiotic susceptibility test comprise a phage-based antibiotic resistance test or a phage-based antibiotic susceptibility test. Preferably, the phage-based identification process is a calorimetric test. Preferably, the method further comprises, if bacteria are determined to be present in the automatic blood culturing apparatus, carrying out a Gram stain analysis on the combined sample.

In another embodiment, the invention provides a method of identifying a bacterium present in a sample of blood, the method comprising: (a) combining at least a first part the sample of blood and a nutrient medium suitable for the growth of bacteria to produce a bacteria growth sample; (b) inserting at least a first portion of the bacterial growth sample in an automatic blood culturing apparatus to determine if bacteria are present in the blood sample; and (c) while the blood culturing apparatus is determining if bacteria are present in the blood sample, performing a phage-based microorganism identification process to identify any bacteria present in the blood. Preferably, the performing a phage-based microorganism identification process is done on a second portion of the bacteria growth sample. Preferably, the combining comprises combining a second part of the sample of blood with an amount of phage capable of attaching to or infecting the bactrium to create a phage-exposed sample, and the performing comprises carrying out the phage-based microorganism identification process on the phage-exposed sample. Preferably, the combining includes combining a nutrient medium suitable for growth of bacteria with the second part or the blood sample. Preferably, the method further comprises dividing the phage-exposed sample into a first fraction and a second fraction; and the performing comprises carrying out the phage-based identification process on the first fraction and conducting an antibiotic resistance test or antibiotic susceptibility test on the second fraction.

In still another aspect, the invention provides a method of determining if a microorganism present in a sample is resistant to or susceptible to an antibiotic, the method comprising: (a) performing a test on the sample capable of detecting the presence of a microorganism in the sample without identifying the microorganism; (b) if the performing does not detect the presence of a microorganism, declaring a negative result; and (c) if the performing detects the presence of a microorganism in the sample, determining if the microorganism is resistant to or susceptible to an antibiotic using a phage-based antibiotic resistance or susceptibility process. Preferably, the performing comprises an automatic blood culturing process.

In yet another aspect, the invention provides a method of determining if a microorganism present in a sample is resistant to or susceptible to an antibiotic, the method comprising: (a) performing a test on the sample capable of detecting the presence of a microorganism in the sample without identifying the microorganism; and (b) while the performing is being done, determining if the microorganism is resistant to or susceptible to an antibiotic using a phage-based antibiotic resistance or susceptibility process. Preferably, the performing comprises an automatic blood culturing process.

The invention permits the long experience in conventional processes to detect the presence of a microorganism, such as the conventional blood culturing process, to become a fail-safe mechanism for the yet-to-be-commercially-proven bacteriophage identification process. Numerous other features, objects, and advantages of the invention will become apparent from the following description when read in conjunction with the accompanying drawings.

### BRIEF DESCRIPTION OF THE DRAWINGS

FIG. 1 illustrates an exemplary assay according to the invention in which a microorganism detection test is combined with a phage-based microorganism identification process with the microorganism detection and microorganism identification processes performed in series;

FIG. 2 illustrates another exemplary assay according to the invention in which the microorganism detection and microorganism identification processes are performed in parallel;

FIG. 3 illustrates an exemplary process according to the invention in which a blood culture bacteria detection test is combined with a phage-based microorganism identification test;

FIG. 4 illustrates the preferred process according to the invention in which an automatic blood culture bacteria detection test is combined with a phage-based microorganism identification test;

FIG. 5 illustrates an exemplary antibiotic resistance test or antibiotic susceptibility test according to the invention; and

FIG. 6 shows a side plan view of a lateral flow microorganism detection device according to the invention.

### DETAILED DESCRIPTION OF THE PREFERRED EMBODIMENT

The invention comprises the combination of a microorganism detection apparatus or process with a bacteriophage-based bacteria identification apparatus or process. In this disclosure, “microorganism detection” means that the presence of a microorganism is ascertained without identifying the specific microorganism or microorganisms that are present. “Identification” means that the specific genus, species, or strain of the microorganism is identified. In this disclosure, the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, yeasts and other microscopic living organisms and uses them to replicate itself. Here, “microscopic” means that the largest dimension is one millimeter or less. Bacteriophage are viruses that have evolved in nature to use bacteria as a means of replicating themselves. A phage does this by attaching itself to a bacterium and injecting its DNA into that bacterium, inducing it to replicate the phage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication or amplification in the bacterium, to lysing of the bacterium takes anywhere from tens of minutes to hours, depending on the phage and bacterium in question and the environmental conditions.

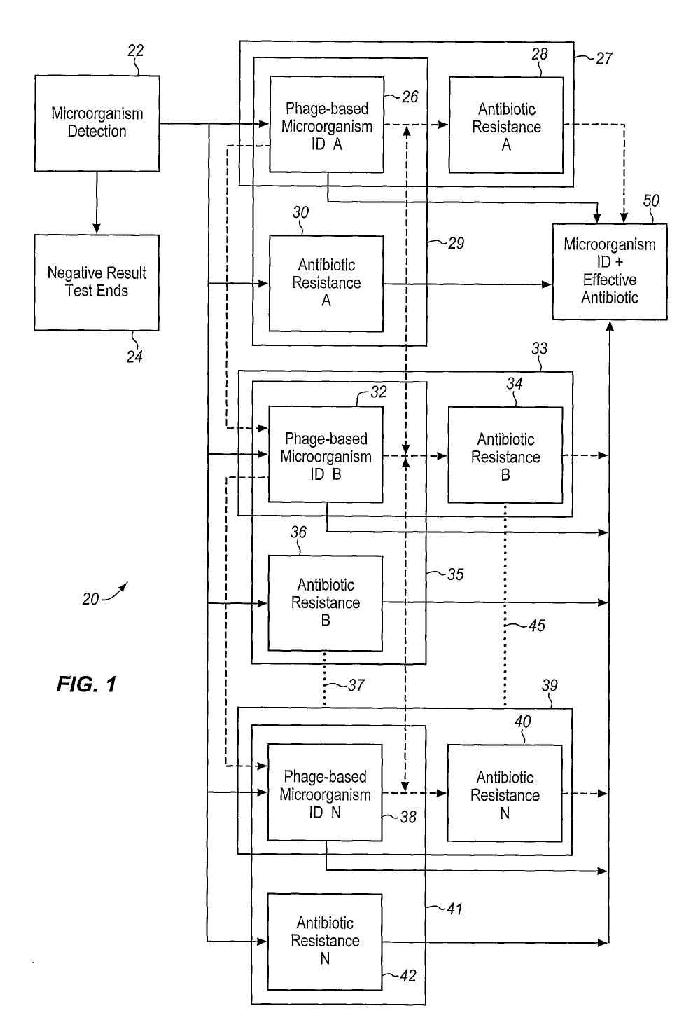

FIG. 1 illustrates several preferred embodiments of the process of the invention. The most preferred embodiment 20 is shown by the solid lines, while optional embodiments are illustrated by the dashed lines. In the most preferred embodiment, the presence of a microorganism is detected at 22. Any one of a wide variety of microorganism detection processes may be used, such as blood culture, autofluorescence, Gram stain, Wright's stain, acridine orange ptl, glucose, dipstick, nitrides-on-silicon chips, microwave resonance cavity, or immunological methods. All of the above methods of detection are know in the art, and thus there is no necessity of detailed description herein. The preferred method is the detection of carbon dioxide produced by most microorganisms, most preferably in an automatic blood culture method. This method is described in more detail below. If the microorganism detection process 22 is negative, that is no microorganism is detected, the test preferably ends at 24. Since most blood culture samples tested for microorganisms are negative samples, this greatly reduces the number of samples on which a phage-based test must be performed, which allows multiple phage-based tests to be performed in a focused and economical manner. It also makes the overall process less dependent on the relatively new phage-based test.

The invention also contemplates that microorganism detection process 22 comprises a plurality of detection processes, such as a combination of two or more of the methods mentioned above. For example, one detection process may ascertain that a microorganism is present, and a second may narrow the possibilities of which microorganism is present, without specifically identifying it. Or one detection process may ascertain within a 70% certainty that a microorganism is not present, and a second may increase the certainty to 95%. It is preferable that when a negative result is found, that the certainty that the test is negative be 95% or greater, more preferably, 99% or greater, and most preferably 99.5% or greater.

If the microorganism detection process is positive, the process 20 proceeds to aphage-based microorganism identification (ID) process 26. Phage-based microorganism ID process 26 is designed to identify a specific microorganism A in the sample. If microorganism A is present in the sample, then the result of the phage-based microorganism ID process 26 is positive. If microorganism A is not present, then the result is negative. Any phage-based microorganism ID process may be used in the process of the invention. For example, it may use a phage amplification process, such as a process described in United States Patent Publication No. 2005/0003346 entitled “Apparatus and Method For Detecting Microscopic Living Organisms Using Bacteriophage”. Or, it may use a process of attaching to a microorganism, such as described in PCT patent application No. PCT/US06/12371 entitled “Apparatus And Method For Detecting Microorganisms Using Flagged Bacteriophage”. Any other phage-based identification process may also be used.

Preferably, antibiotic resistance test 30 proceeds in parallel with phage-based microorganism ID process 26, that is, at the same time. Any antibiotic resistance test known to those skilled in the art may be used in the process of the invention. However, if the original sample may contain multiple microorganisms, then antibiotic resistance test 30 should specifically test only a single microorganism. In a preferred method of the invention taught herein, antibiotic resistance test 30 is a phage based process similar to or identical with phage-based microorganism ID process 26, but performed in the presence of a predetermined concentration of a selected antibiotic.

Antibiotic resistance test 30 is used to determine whether or not microorganism A, if present in the sample, is resistant to a specific antibiotic at a specific concentration. If it is present and resistant, then the result of antibiotic resistance test 30 is positive. If not, the result of test 30 is negative.

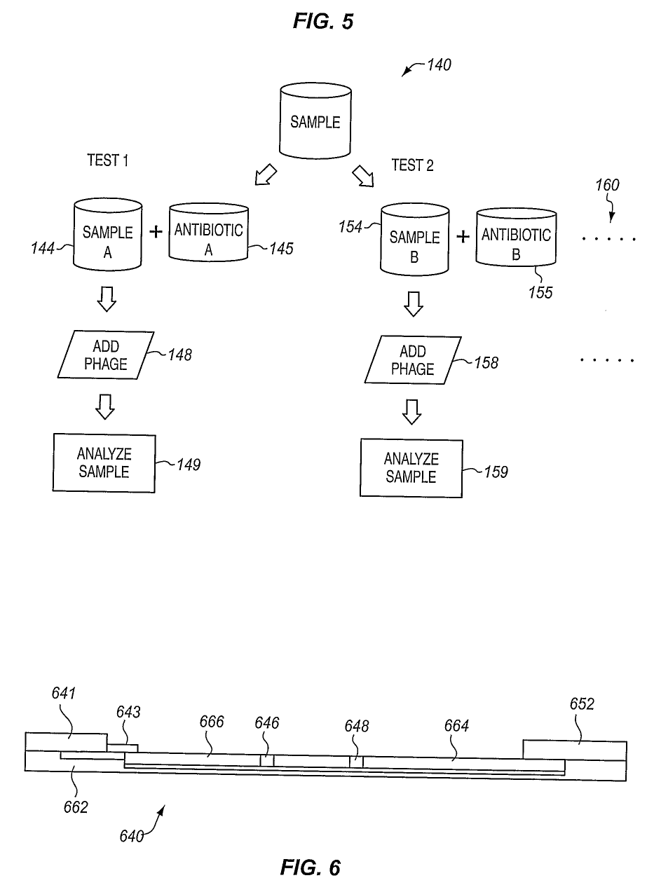

Preferably, a plurality 26, 32, and 38 of phage-based ID processes are performed in parallel, each involving a different phage or combination of phages and different target microorganisms. Preferably, a plurality 30, 36, and 42 of antibiotic resistance tests are also performed in parallel. Preferably, each of the antibiotic resistance tests 30, 36 and 42 represent a plurality of tests, each with a different antibiotic and/or with different antibiotic concentrations, as indicated in FIG. 5. Generally, as indicted in FIG. 5, the number of antibiotic resistance tests that are performed may be different than the number of ID processes. In addition, the dots 37 indicate that both additional phage-based ID processes and antibiotic resistance tests may be performed.

Clinically, it is often more valuable to determine the susceptibility of a microorganism to an antibiotic rather than its resistance. Armed with this information, a physician knows that a specific antibiotic at a specific dosage can be used to successfully treat a patient. Phage-based microorganism ID process 26 can be used together with antibiotic resistance test 30 to determine the susceptibility of microorganism A, if present in the sample, to a given concentration of antibiotic. Together, process 26 and test 30 comprise antibiotic susceptibility test 29 as indicated in FIG. 1. The result of antibiotic susceptibility test 30 is positive if a) phage-based microorganism ID process 26 gives a positive result, indicating the presence of microorganism A in the sample, and b) antibiotic resistance test 30 gives a negative result indicating that microorganism A is not resistant to the tested antibiotic concentration. The result of antibiotic susceptibility test 30 is negative if a) phage-based microorganism ID process 26 gives a positive result, indicating the presence of microorganism A in the sample, and b) antibiotic resistance test 30 gives a positive result indicating that microorganism A is resistant to the tested antibiotic concentration. Preferably, a plurality 29, 35, and 41 of antibiotic susceptibility tests are performed in parallel. Preferably, each of the antibiotic susceptibility tests 29, 35 and 41 represent a plurality of tests, each with a different antibiotic and/or with different antibiotic concentrations.

When the phage-based microorganism ID processes A through N and the antibiotic susceptibility tests A through N are completed, the microorganism(s) is identified and an effective antibiotic(s) and with effective dosage(s) at 50.

Alternatively, the phage-based microorganism ID process 26 and the antibiotic resistance test 28 are performed in series; that is, sequentially, as shown by the dashed lines in FIG. 1. ID process 26 and antibiotic resistance test, taken together, comprise antibiotic susceptibility test 27. Again, there are preferably a plurality of microorganism identification processes, 26, 32, and 38; a plurality of antibiotic resistance tests 28, 34 and 40; and a plurality of antibiotic susceptibility tests 27, 33, and 39. Again, each of the antibiotic resistance studies 28, 34, and 40 represent a plurality of tests, each with a different antibiotic and/or antibiotic concentration. The dots 37 and 45 indicate that additional phage-based microorganism ID processes and antibiotic resistance or susceptibility tests may be performed. Again, when the phage-based microorganism ID processes A through N and the antibiotic susceptibility studies A through N are completed, the microorganism(s) is identified and an effective antibiotic(s) and dosage(s) are determined at 50.

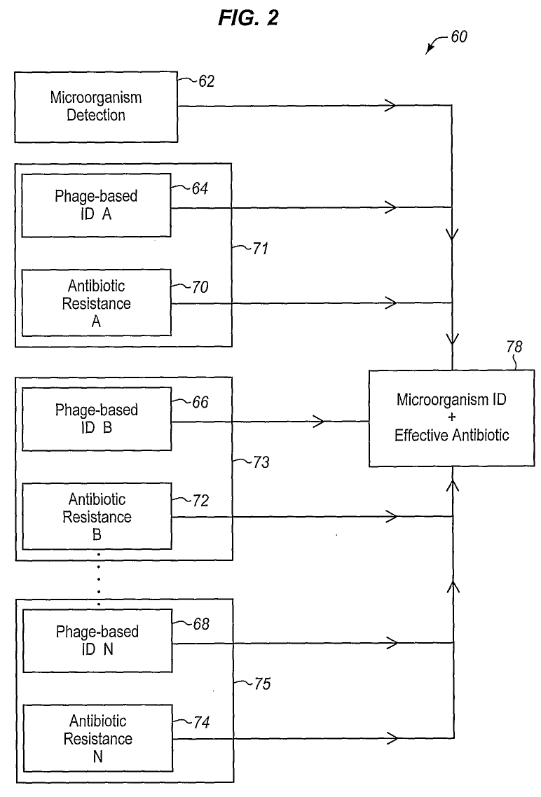

FIG. 1 illustrates an embodiment of the inventive process in which the microorganism detection 22 and the bacteriophage-based ID process, such as 26, are performed in series, that is, with the bacteriophage-based ID process following the microorganism detection. FIG. 2 illustrates an embodiment 60 of the inventive process in which the blood microorganism detection 62 and the bacteriophage-based ID process 64 are performed in parallel, that is, with the bacteriophage-based ID process performed while the detection process is being preformed. This embodiment may be preferred in situations where, prior to the presence of a microorganism being definitively detected, there are indications that a patient has an especially acute infection or infection by a particularly virulent pathogen such as methicillin resistant Staph aureus (MRSA) is suspected. In such cases, quickly determining the identity of selected microorganisms is of greater consequence, thus it would be appropriate to start the identification process as soon as possible. Again, in this embodiment a plurality of phage-based microorganism ID processes 64, 66, 68, are performed at the same time. Again, a plurality of antibiotic resistance tests 70, 72, and 74 are also performed in parallel. Again, ID process 64 and antibiotic resistance test 70 together comprise antibiotic susceptibility test 71, ID process 66 and resistance test 72 comprise susceptibility test 73, and so on through antibiotic susceptibility test 75. The microorganism detection 62 and the phage-based ID process A 64, are preferably performed in separate subsamples of the sample to be tested, but alternatively may be performed in the same subsample. When the phage-based microorganism ID processes A through N and the phage-based susceptibility studies A through N are completed, the microorganism(s) is identified and an effective antibiotic(s) and dosage(s) are determined at 78.

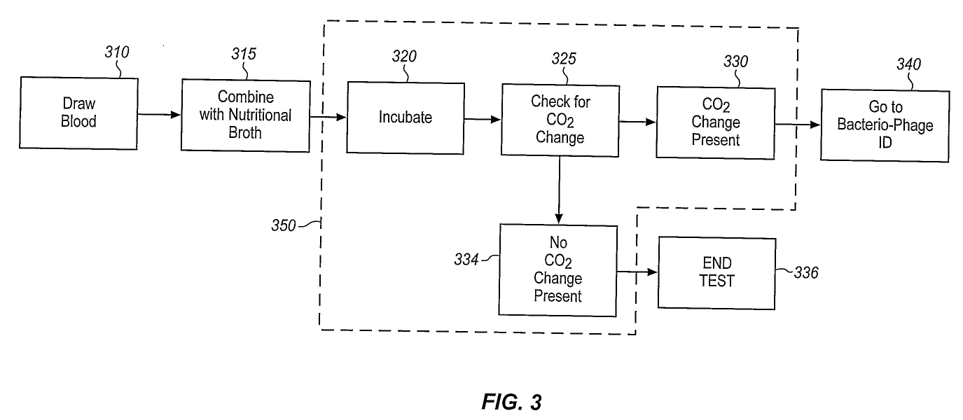

Referring to FIG. 3, an example of the microorganism detection processes 22 and 62 is shown. The preferred microorganism detection process when the sample is a blood sample is an automatic blood culture process 300. In such a process, blood is drawn at 310 and combined 315 in a bottle or blood collection tube with a nutritional broth suitable for serving as a growth medium for bacteria. The combined sample is placed in a blood culture machine 350 where it is incubated 320 and regularly checked 325 to determine if bacteria are present. Blood culture machine 350 generally relies on changing CO₂ (carbon dioxide) concentration to determine the presence of “microbial growth” within the culture. Here, microbial growth is put in quotation marks because there are a number of different possible sources of carbon dioxide, including growth of bacteria, yeasts, molds, white blood cell death, etc. If the blood culture machine 350 determines that the CO₂ concentration is changing 330 the detection is declared positive, and the process proceeds to the bacteriophage-based ID process 340. If the blood culture machine 350 determines 334 that the CO₂ concentration does not change over a predetermined period of time, the test is considered negative and is ended 336. The ID process may be performed on the same sample as the one on which the process to determine the presence of bacteria is done. Or, the bacteria determination process may be done on a first portion of the combined sample, and the ID process performed on a second portion. As another alternative, a first part of the blood sample may be combined with the nutritional broth and the presence of bacteria determined with this first combined sample, while a second part of the blood sample is combined with a second portion of the nutritional broth and the ID process performed on this second combined sample. Other variations may be designed by those skilled in the art. While the automated blood process system 300 described herein is preferred, any conventional blood culture process may be used. An automatic blood culture process and apparatus is described in U.S. Pat. No. 5,817,508 508 issued to Klaus W. Berndt on Oct. 6, 1998, which is incorporated by reference to the same extent as though fully disclosed herein. The blood culture process 300 is known in the art, and will not be described in more detail herein.

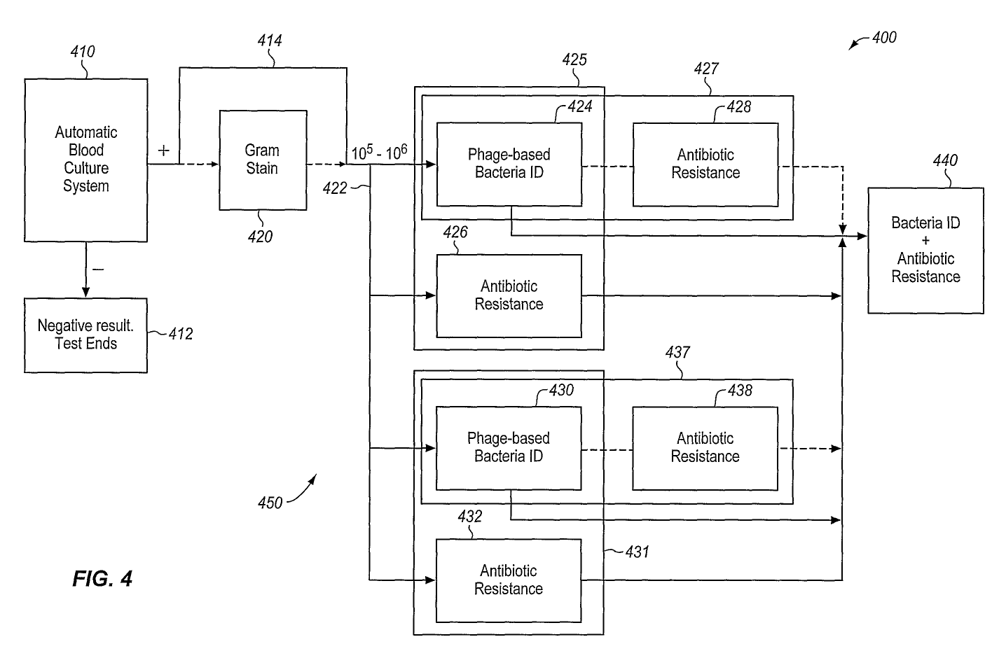

FIG. 4 illustrates a preferred system and process 400 according to the invention which incorporates an automatic blood culture system 410 with a phage-based microorganism ID and antibiotic susceptibility system 450. In the automatic blood culture process, the collection tubes containing the blood sample in a growth medium are placed into an automated blood culture system 410 (i.e., Bactec, Becton, Dickinson, & Company; BacT/Alert, bioMerieux) that performs the functions 350 of FIG. 3. The blood collection tube containing the sample in the nutritional broth is incubated 320 and regularly checked 325 to determine if bacteria are present. If the blood culture result is negative, the test ends 412. If the blood culture result is positive, the process usually proceeds along branch 414. A positive automatic blood culture test generally results in a sample with approximately 10⁵ or more bacteria per milliliter (mL) as shown at junction 422. This sample is generally divided into a plurality of subsamples, upon which a plurality of phage-based bacteria ID processes, 424, 430 are carried out simultaneously, each employing a different variety of bacteriophage. The phage-based ID microorganism process will be described in more detail below. Generally, an antibiotic resistance test 426, 432 is performed in parallel with each microorganism ID process 424, 430. ID processes 424 and 430 when combined with antibiotic resistance tests 426 and 432 respectively comprise antibiotic susceptibility tests 425 and 431 as shown in FIG. 4. As indicated above, preferably, each antibiotic resistance test 426, 432 comprises a plurality of tests, each with a different antibiotic and/or with differing antibiotic concentrations. However, the invention also contemplates that a antibiotic resistance test 428, 438 may optionally be performed in series with the phage-based microorganism ID process 424, 430. ID processes 424 and 430 when combined with antibiotic resistance tests 428 and 438 respectively comprise antibiotic susceptibility tests 427 and 437 as shown in FIG. 4. If the antibiotic resistance tests 428, 438 are performed in series, the parallel tests 426 and 432 are not usually performed. As another option, a second bacteria detection process 420 may be performed between the blood culture process 410 and the phage-based microorganism ID processes and antibiotic resistance test or antibiotic susceptibility tests 450. In the preferred alternative, the second bacteria detection process 420 is a Gram stain test. Performing a Gram stain test 420 may assist in narrowing the range of bacteria that could be present, and thus reduce the number of phage-based ID processes 424 . . . 430 and antibiotic resistance test or antibiotic susceptibility tests 426 . . . 432 that need to be performed. The result 440 of the tests 410, 424, 425, 426 (or 427 and 428), 430, 431, and 432 (or 437 and 438) is that both the type of bacteria causing the infection and the antibiotic and dosage that will best kill or retard the growth of the bacteria are identified at 440.

FIG. 5 illustrates the preferred antibiotic resistance tests 28, 30, 70, 426, etc, used herein, that is, a method 140 by which any phage-based test can be used to determine if the bacterium present is resistant to one or more antibiotics. A sample 142 that contains the target bacterium is divided into a first Sample A, indicated by 144, a second Sample B, indicted by 154, and as many additional samples, as indicted by the dots 160, that are needed to test all of antibiotics to be tested. A first antibiotic 145 is added to Sample A, a second antibiotic (or the same antibiotic at a different concentration) 155 is added to Sample B, and other antibiotics (or concentrations) are added to the samples indicted at 160. The target bacteria in the samples are killed or growth is retarded if they are not resistant to the antibiotic in the sample. After a suitable time for the antibiotic to act on the bacteria, a quantity of phage is added at 148, 158, etc. The invention also contemplates that the bacteriophage and antibiotic can be added at the same time. In the processes in which the antibiotic resistance tests are performed in parallel with the phage-based microorganism ID process, this will generally be preferred. In any case, after the bacteriophage is added, samples A and B etc. are analyzed after a predetermined period of time at 149 and 159 etc. to detect the presence of viable target bacteria in each. Any bacteriophage detection method, such as the methods mentioned in this disclosure, can be used for these analyses. If bacteria are found to be present, or if the bacterial concentration has increased, it indicates that the bacterium is resistant to the antibiotic. The degree of resistance can be determined by testing different antibiotic concentrations. To screen for the antibiotic resistance of a group of antibiotics simultaneously, then all of the antibiotics of interest are added to one sample and analyzing for the target bacterium. If the target bacterium is detected in the antibiotic treated sample, or if the target bacteria has increased, it indicates that the target bacterium in the sample is resistant to the group of antibiotics.

We turn now to the details of the phage-based microorganism ID processes, 26, 64, 424 etc. and the phage analysis portions 149, 159, etc. of the antibiotic resistance tests 28, 30, 70, 426, etc. Any phage identification method or apparatus that detects phage or some biomarker associated with the phage when a specific microorganism is present can be used in the invention. Preferred methods are immunoassay methods utilizing antibody-binding events to produce detectable signals including ELISA, western blots, radioimmunoassay, immunoflouresence, lateral flow immunochromatography (LFI), and the use of a SILAS surface which changes color as a detection indicator. Other methods are aptamer-based assays, mass spectrometry, such as matrix-assisted laser desorption/ionization with time-of-flight mass spectrometry (MALDI-TOF-MS), referred to herein as MALDI, flow and cytometry. One immunoassay method, LFI, is discussed in detail below in connection with FIG. 6 A cross-sectional view of the lateral flow strip 40 is shown in FIG. 6. The lateral flow strip 640 preferably includes a sample application pad 641, a conjugate pad 643, a substrate 6 64 in which a detection line 646 and an internal control line 648 are formed, and an absorbent pad 652, all mounted on a backing 662, which preferably is plastic. The substrate 664 is preferably a porous mesh or membrane. It is made by forming lines 643, 646, and optionally line 648, on a long sheet of said substrate, then cutting the substrate in a direction perpendicular to the lines to form a plurality of substrates 664. The conjugate pad 643 contains beads each of which has been conjugated to a first antibody forming first antibody-bead conjugates. The first antibody selectively binds to the phage in the test sample. Detection line 646 and control line 648 are both reagent lines and each form an immobilization zone; that is, they contain a material that interacts in an appropriate way with the bacteriophage or other biological marker. In the preferred embodiment, the interaction is one that immobilizes the bacteriophage or other biological marker. Detection line 646 preferably comprises immobilized second antibodies, with antibody line 646 perpendicular to the direction of flow along the strip, and being dense enough to capture a significant portion of the phage in the flow. The second antibody also binds specifically to the phage. The first antibody and the second antibody may or may not be identical. Either may be polyclonal or monoclonal antibodies. Optionally, strip 640 may include a second reagent line 48 including a third antibody. The third antibody may or may not be identical to one or more of the first and second antibodies. Second reagent line 648 may serve as an internal control zone to test if the assay functioned properly.

One or more drops of a test sample are added to the sample pad. The test sample preferably contains parent phage as well as progeny phage if the target bacterium was present in the original raw sample. The test sample flows along the lateral flow strip 640 toward the absorbent pad 652 at the opposite end of the strip. As the phage particles flow along the conjugate pad toward the membrane, they pick up one or more of the first antibody-bead conjugates forming phage-bead complexes. As the phage-bead complexes move over row 646 of second antibodies, they form an immobilized and concentrated first antibody-bead-phage-second antibody complex. If enough phage-bead complexes bind to the row 646 of immobilized second antibodies, a line becomes detectable. The detectability of the line depends on the type of bead complex. As known in the art, antibodies may be conjugated with a color latex, gold particles, or a fluorescent magnetic, paramagnetic, superparamagnetic, or supermagnetic marker, as well as other markers, and may be detected either visually or otherwise as a color, or by other suitable indicator. A line indicates that the target microorganism(s) were present in the raw sample. If no line is formed, then the target microorganisms were not present in the raw sample or were present in concentrations too low to be detected with the lateral flow strip 640. For this test to work reliably, the concentration of parent phage added to the raw sample should be low enough such that the parent phage alone are not numerous enough to produce a visible line on the lateral flow strip. The antibody-bead conjugates are color moderators that are designed to interact with the bacteriophage or a biological substance associated with the bacteriophage. When they are immobilized in the immobilization zone 646, they cause the immobilization zone to change color. A more complete description of the lateral flow strip and process are given in United States Patent Application Publication No. 2005/0003346 published Jan. 6, 2005, which is incorporated herein by reference to the same extent as though fully disclosed herein.

Many other phage-based methods and apparatus may be used to identify the microorganism and/or to determine the antibiotic resistance test or antibiotic susceptibility, i.e., used or partially used in processes 26, 27, 28, 29, 30, 64, 70, 71, 424, 425, 426, 427, and 428426, etc. Examples of such processes are disclosed in the following publications:

### United States Patents:

U.S. Pat. No. 4,104,126 issued Aug. 1, 1978 to David M. Young
U.S. Pat. No. 4,797,363 issued Jan. 10, 1989 to Teodorescu et al.
U.S. Pat. No. 4,861,709 issued Aug. 29, 1989 to Ulitzur et al.
U.S. Pat. No. 5,085,982 issued Feb. 4, 1992 to Douglas H. Keith
U.S. Pat. No. 5,168,037 issued Dec. 1, 1992 to Entis et al.
U.S. Pat. No. 5,498,525 issued Mar. 12, 1996 to Rees et al.
U.S. Pat. No. 5,656,424 issued Aug. 12, 1997 to Jurgensen et al.
U.S. Pat. No. 5,679,510 issued Oct. 21, 1997 to Ray et al.
U.S. Pat. No. 5,723,330 issued Mar. 3, 1998 to Rees et al.
U.S. Pat. No. 5,824,468 issued Oct. 20, 1998 to Scherer et al.
U.S. Pat. No. 5,888,725 issued Mar. 30, 1999 to Michael F. Sanders
U.S. Pat. No. 5,914,240 issued Jun. 22, 1999 to Michael F. Sanders
U.S. Pat. No. 5,958,675 issued Sep. 28, 1999 to Wicks et al.
U.S. Pat. No. 5,985,596 issued Nov. 16, 1999 to Stuart Mark Wilson
U.S. Pat. No. 6,090,541 issued Jul. 18, 2000 to Wicks et al.
U.S. Pat. No. 6,265,169 B1 issued Jul. 24, 2001 to Cortese et al.
U.S. Pat. No. 6,300,061 B1 issued Oct. 9, 2001 to Jacobs, Jr. et al.
U.S. Pat. No. 6,355,445 B2 issued Mar. 12, 2002 to Cherwonogrodzky et al.
U.S. Pat. No. 6,428,976 B1 issued Aug. 6, 2002 to Chang et al.
U.S. Pat. No. 6,436,652 B1 issued Aug. 20, 2002 to Cherwonogrodzky et al.
U.S. Pat. No. 6,436,661 B1 issued Aug. 20, 2002 to Adams et al.
U.S. Pat. No. 6,461,833 B1 issued Oct. 8, 2002 to Stuart Mark Wilson
U.S. Pat. No. 6,524,809 B1 issued Feb. 25, 2003 to Stuart Mark Wilson
U.S. Pat. No. 6,544,729 B2 issued Apr. 8, 2003 to Sayler et al.
U.S. Pat. No. 6,555,312 B1 issued Apr. 29, 2003 to Hiroshi Nakayama

### United States Published Applications:

2002/0127547 A1 published Sep. 12, 2002 by Stefan Miller
2004/0121403 A1 published Jun. 24, 2004 by Stefan Miller
2004/0137430 A1 published Jul. 15, 2004 by Anderson et al.
2005/0003346 A1 published Jan. 6, 2005 by Voorhees et al.

### Foreign Patent Publications:

EPO 0 439 354 A3 published Jul. 31, 1991 by Bittner et al.
WO 94/06931 published Mar. 31, 1994 by Michael Frederick Sanders
EPO 1 300 082 A2 published Apr. 9, 2003 by Michael John Gasson
WO 03/087772 A2 published Oct. 23, 2003 by Madonna et al.

### Other Publications:

Favrin et al., “Development and Optimization of a Novel Immunomagnetic Separation-Bacteriophage Assay for Detection of Salmonella enterica Serovar Enteritidis in Broth”, Applied and Environmental Microbiology, January 2001, pp. 217-224, Volume 67, No. 1.
All of the forgoing publications are hereby incorporated by reference to the same extent as though fully disclosed herein. Any other bacteriophage-based process may be used as well.

A feature of the invention is the synergistic nature of the combination of the detection process 22, 62, 300 or apparatus 350 and the phage-based microorganism ID process. A reason why a commercially available phage-based ID process was not developed prior to the present disclosure, is that to be most effective, phage-based ID processes to date require the presence of a large number of bacteria. However, the invention recognizes that upon the completion of the typical detection process, such as the blood culturing process 410, 10⁵ or more bacteria will be present. The invention recognizes that this is enough bacteria for the phage-based ID process to proceed quickly and effectively. Generally, the blood culturing process 510 takes six to eighteen hours to complete. Conventional bacteria culturing processes that were used in combination with prior art blood-culturing tests generally take twelve to thirty-six hours to complete. Conventional antibiotic susceptibility tests that were used with prior art blood culturing tests take anywhere from twenty-four to thirty-six hours to complete. Thus, conventional blood culture tests took anywhere from forty-two to ninety hours to arrive at a complete result identifying the bacteria and the best antibiotic to use against the bacteria. Of this time, thirty-six to seventy-two hours after completion of the blood culture were required to identify the bacteria and determine the best antibiotic. In comparison, the blood culturing test system according to the invention takes only one to six hours after completion of the blood culture.

Another feature of the invention is that the phage-based microorganism ID process distinguishes between live and dead bacteria. This is essential for antibiotic resistance test or antibiotic susceptibility tests, food applications where the food has been irradiated, or any other application where dead bacteria may be present. Thus, the invention provides significant advantages over other relatively fast ID tests, such as nucleic acid-based technologies (PCR etc.), immunological tests, aptamers, etc., in which it is impossible or difficult to distinguish between live and dead bacteria.

Another feature of the invention is that the phage-based microorganism ID process is simpler and less expensive than other bacteria identification tests, such as molecular methods. This permits a blood culture system that remains relatively inexpensive, while at the same time being significantly speeded up. A further feature of the invention is that the antibiotic resistance subprocess 28, 30, 70, 428, 426 etc. is also simple and can follow protocols that are similar to conventional antibiotic resistance test or antibiotic susceptibility processes, thus little training is required.

Another feature of the invention is that the invention recognizes that detection process, such as the blood culturing process, acts as a good prescreening method for a phage-based microorganism ID processes. In the blood culturing process, approximately 93% of the blood samples processed produce a negative result. Thus, the phage-based assay needs to be applied to only about seven percent of the total blood samples tested, and it is known that most of these samples do contain bacteria. There has been described a microorganism detection method which is specific to a selected organism, sensitive, simple, fast, and/or economical, and having numerous novel features. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by microorganism detection apparatus and methods described.

We claim:

1. A method of identifying a microorganism present in a sample, said method comprising:
(a) performing a test on said sample capable of detecting the presence of a microorganism in said sample without identifying said microorganism;
(b) if said performing does not detect the presence of a microorganism, declaring a negative result; and
(c) if said performing detects the presence of a microorganism in said sample, identifying the microorganism present in said sample using a phage-based microorganism identification process.

2. A method as in claim 1 and further comprising conducting an antibiotic resistance test or antibiotic susceptibility test on said sample.

3. A method as in claim 2 wherein said identifying is performed on a first sample, said conducting comprises conducting an antibiotic resistance test on a second sample, and said antibiotic susceptibility test comprises: said identifying said microorganism in said first sample and said conducting said antibiotic resistance test on said second sample.

4. A method as in claim 2 wherein said conducting comprises conducting a plurality of antibiotic resistance tests on a plurality of samples, each said antibiotic resistance test utilizing a different antibiotic or a different concentration of antibiotic.

5. A method as in claim 2 wherein said antibiotic resistance test or said antibiotic susceptibility test comprise a phage-based antibiotic resistance test or a phage-based antibiotic susceptibility test.

6. A method as in claim 1 wherein said identifying comprises a calorimetric test.

7. A method as in claim 1 wherein said performing comprises carrying out a plurality of different tests capable of detecting the presence of a microorganism in said sample.

8. A method as in claim 7 wherein said microorganism is a bacteria and said plurality of different tests are selected from the group consisting of blood culture, autofluorescence, Gram stain, Wright's stain, acridine orange ptl, glucose, dipstick, nitrides-on-silicon chips, microwave resonance cavity, or immunological methods.

9. A method as in claim 8 wherein said plurality of tests comprise an automatic blood culture test and a Gram stain test.

10. A method as in claim 1 wherein said phage-based microorganism identification process comprises one or more tests selected from the group consisting of: immunoassay methods, aptamer-based assays, mass spectrometry, including MALDI, and flow cytometry.

11. A method as in claim 10 wherein said immunoassy methods are selected from the group consisting of ELISA, western blots, radioimmunoassay, immunoflouresence, lateral flow immunochromatography (LFI), and a test using a SILAS surface.

12. A method as in claim 10 wherein said microorganism is a bacteria and said performing comprises one or more methods selected from the group consisting of blood culture, autofluorescence, Gram stain, Wright's stain, acridine orange ptl, glucose, dipstick, nitrides-on-silicon chips, microwave resonance cavity, or immunological methods.

13. A method of identifying a microorganism present in a sample, said method comprising:
(a) performing a test on said sample capable of detecting the presence of a microorganism in said sample without identifying said microorganism; and
(b) while said performing is being done, identifying the microorganism present in said sample using a phage-based microorganism identification process.

14. A method as in claim 13 and further comprising, if said performing does not detect the presence of a microorganism declaring a negative result.

15. A method of identifying a bacterium present in a sample of blood, said method comprising:
(a) combining said sample of blood and a nutrient medium suitable for the growth of bacteria;
(b) inserting said combined sample in an automatic blood culturing apparatus to determine if bacteria are present in said blood sample; and
(c) if bacteria are determined to be present in said automatic blood culturing apparatus, performing a phage-based microorganism identification process on said combined sample to identify the bacteria present in said blood.

16. A method as in claim 15 and further comprising conducting an antibiotic resistance test or antibiotic susceptibility test on said combined sample.

17. A method as in claim 16 wherein said antibiotic resistance test or said antibiotic susceptibility test comprise a phage-based antibiotic resistance test or a phage-based antibiotic susceptibility test

18. A method as in claim 15 wherein said phage-based identification process is a calorimetric test.

19. A method as in claim 15 and further comprising, if bacteria are determined to be present in said automatic blood culturing apparatus, carrying out a Gram stain analysis on said combined sample.

20. A method of identifying a bacterium present in a sample of blood, said method comprising:
(a) combining at least a first part said sample of blood and a nutrient medium suitable for the growth of bacteria to produce a bacteria growth sample;
(b) inserting at least a first portion of said bacterial growth sample in an automatic blood culturing apparatus to determine if bacteria are present in said blood sample; and
(c) while said blood culturing apparatus is determining if bacteria are present in said blood sample, performing a phage-based microorganism identification process to identify any bacteria present in said blood.

21. A method as in claim 20 wherein said performing a phage-based microorganism identification process is done on a second portion of said bacteria growth sample.

22. A method as in claim 20 wherein said combining comprises combining a second part of said sample of blood with an amount of phage capable of attaching to or infecting said bactrium to create a phage-exposed sample, and said performing comprises carrying out said phage-based microorganism identification process on said phage-exposed sample.

23. A method as in claim 22 wherein said combining includes combining a nutrient medium suitable for growth of bacteria with said second part or said blood sample.

24. A method as in claim 23 and further comprising dividing said phage-exposed sample into a first fraction and a second fraction; and said performing comprises carrying out said phage-based identification process on said first fraction and conducting an antibiotic resistance test or antibiotic susceptibility test on said second fraction.

25. A method of determining if a microorganism present in a sample is resistant to or susceptible to an antibiotic, said method comprising:
(a) performing a test on said sample capable of detecting the presence of a microorganism in said sample without identifying said microorganism;
(b) if said performing does not detect the presence of a microorganism, declaring a negative result; and
(c) if said performing detects the presence of a microorganism in said sample, determining if said microorganism is resistant to or susceptible to an antibiotic using a phage-based antibiotic resistance or susceptibility process.

26. A method as in claim 25 wherein said performing comprises an automatic blood culturing process.

27. A method of determining if a microorganism present in a sample is resistant to or susceptible to an antibiotic, said method comprising:
(a) performing a test on said sample capable of detecting the presence of a microorganism in said sample without identifying said microorganism; and
(b) while said performing is being done, determining if said microorganism is resistant to or susceptible to an antibiotic using a phage-based antibiotic resistance or susceptibility process.

28. A method as in claim 27 wherein said performing comprises an automatic blood culturing process.
